(19) United States
(12) Patent Application Publication
(10) Pub. No.: US 2026/0091168 A1
(43) Pub. Date: Apr. 2, 2026

(54) SYRINGES FOR INFUSION PUMPS

(71) Applicant: ICU Medical, Inc., San Clemente, CA (US)

(72) Inventors: James Jacobson, Lindenhurst, IL (US); Robert Butterfield, Poway, CA (US); Robert Hlinsky, Glenview, IL (US); Marshall Fryman, Libertyville, IL (US); Ross Spence, Morrisville, NC (US)

(21) Appl. No.: 19/110,450

(22) PCT Filed: Sep. 18, 2023
(86) PCT No.: PCT/US2023/074487

§ 371 Date: Mar. 11, 2025

Related U.S. Application Data
(60) Provisional application No. 63/407,512, filed on Sep. 16, 2022.

(51) Int. Cl. A61M 5/145; A61M 5/31; A61M 5/315

(52) U.S. Cl. CPC A61M 5/1452; A61M 5/3129; A61M 5/31513; A61M 2005/3131

(57) ABSTRACT

Embodiments of syringes and components thereof described herein provide reduced compliance from flexing or deformation, of not only the syringe barrel but also of the syringe plunger rod and plunger seal. Embodiments of syringes and components thereof described herein also provide improved sealing between the barrel and the syringe plunger rod, and improved resistance to pressure. Embodiments of syringes and components thereof described herein are advantageously suited for use with an infusion pump which may subject syringes to many start/stop operations over a period of hours, sometimes at very low flow rates.

### CROSS REFERENCE TO RELATED APPLICATIONS

The present application claims the benefit of Provisional Application No. 63/407,512, filed Sep. 16, 2022, the disclosure of which is hereby incorporated by reference.

### FIELD

The present disclosure generally pertains to syringes. More particularly, the present disclosure pertains to syringes for use with infusion pumps.

### BACKGROUND

Infusion pumps are extremely useful medical devices for providing prescribed fluids, drugs, and other therapies (collectively, “infusates”) to patients in controlled amounts. For example, medications such as antibiotics, chemotherapy drugs, vasoactives, insulin, blood products, and pain relievers are commonly delivered to patients via an infusion pump, as are nutrients and other supplements. Infusion pumps have been used in hospitals, nursing homes, and in other short-term and long-term medical facilities, as well as for in-home care. Infusion pumps can be particularly useful for the delivery of medical therapies requiring an extended period of time for their administration. There are many types of infusion pumps, including large volume, patient-controlled analgesia (PCA), elastomeric, syringe (syringe driver), enteral, and insulin pumps. Infusion pumps are typically useful in various routes of medication delivery, including intravenously, intra-arterially, subcutaneously, intraperitoneally, intraosseous, intraportal, in close proximity to nerves, and into an intraoperative site, epidural space or subarachnoid space.

Syringe pumps have a number of desirable characteristics, and are generally perceived as the most precise and accurate acute care infusion pumps available. Syringe pumps may support lower flow rates than large volume pumps or ambulatory pumps, sometimes as low as 0.01 milliliters/hour (with appropriately-sized small syringes). Unlike large volume and ambulatory pumps that utilize proprietary or dedicated consumables, syringe pumps typically accommodate wide ranges of commonly used or “off-the-shelf” syringe brands and sizes that are typically coupled with non-proprietary extension sets for delivering infusates to patients.

However, traditional off-the-shelf syringes are not specifically designed for use in syringe pumps, as a very small percent of worldwide syringes are used with pumps. Typically off-the-shelf syringes are designed for manual, hand use. But when used with a syringe pump, the pump must identify or otherwise be configured with relevant characteristics of an off-the-shelf syringe for proper operation of the pump. Problems with syringe pump systems can arise from both the pump and the syringe. Some syringe pumps can suffer from performance limitations, particularly at low flow rates (<5 milliliters per hour and particularly below 0.1 mL/hr), including but not limited to long start-up times to reach target flow rates, inconsistent flow profiles during infusate delivery, long times to alarm an occlusion, and risk of inadvertently delivering a bolus or allowing retrograde flow. Further, some off-the-shelf syringes can also contribute to inaccuracies due to syringe compliance (e.g., deformation under pressure), which increases the time for a syringe pump to reach a programmed target flow rate, the time for a syringe pump to recognize an occlusion, and the volume of unintended boluses or no-flow period due to height changes to a running syringe pump, among other challenges. Further inaccuracies may arise from changes in backpressure due to multiple pumps being added to the same infusion line.

Off-the-shelf syringes can characteristically change over time, knowingly or unknowingly, thereby impacting pump performance. Further, off-the-shelf syringes can contribute to flow inconsistency and short-term inaccuracy, particularly with “stick slip” behavior, where flow delivery is discretized in delivery following periods of inadvertent no (or reduced) flow. Some off-the-shelf syringes exhibit high friction forces which, in combination with mechanical compliances of a pump, can be a source of stick-slip behavior and delayed start-of-flow. In one experiment that was conducted, pertaining to the present disclosure, a commercially available fifty milliliter syringe was operated in a syringe pump at a flow rate of 0.5 milliliters per hour, for a duration of eighty-four hours. After nearly thirty hours of nominal flow, a spontaneous onset of stick-slip flow episodes began as depicted in FIG. 1, resulting in inconsistent fluid delivery from the syringe.

While valuable improvements to syringe pump construction, configurations, and operations have been and continue to be made, there remains a need for improvements to syringes themselves for their uses with syringe pumps. The present disclosure addresses these concerns.

### SUMMARY

Embodiments described or otherwise contemplated herein substantially provide the advantages of improving ease of use, operation, accuracy, and patient safety in the delivery of infusates, among other advantages.

In a feature and advantages of embodiments, syringe pumps operating on a syringe according to embodiments described herein feature reduced pump start-up time and reduced syringe swap time. In a feature and advantages of embodiments, time to detection of syringe pump occlusion is decreased. In a feature and advantages of embodiments, completeness of delivery of the infusate dose is increased.

In a feature and advantages of embodiments, flow constancy of medicament delivered from the syringe is improved and delivery anomalies such as stick-slip are reduced. Embodiments of syringes described herein improve both short-term and long-term pump accuracy across the full range of pump operation, decreasing dependence of performance on environmental conditions such as backpressure, temperature, or fluid variations.

In a feature and advantages of embodiments, unintended bolus volume and no flow periods (e.g., due to height change of running syringe pumps) are reduced.

Embodiments of syringes and components thereof described herein provide reduced compliance from flexing or deformation, of not only the syringe barrel but also of the syringe plunger rod and plunger seal. Embodiments of syringes and components thereof described herein also provide improved sealing between the barrel and the syringe plunger rod, and improved resistance to pressure. Embodiments of syringes and components thereof described herein are advantageously suited for use with an infusion pump which may subject syringes to many start/stop operations over a period of hours, sometimes at very low flow rates.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a graph of flow rate over time of a prior art syringe, depicting inconsistent fluid delivery from the syringe.

FIG. 2 is a perspective view of an example syringe pump for use with embodiments of the disclosure.

FIG. 3 is a perspective exploded view of a syringe, according to an embodiment of the disclosure.

FIG. 4 is a perspective view of a plunger rod, according to an embodiment of the disclosure.

FIG. 5 is a perspective view of a plunger rod sealing element, according to an embodiment of the disclosure.

FIG. 6 is a perspective view of a plunger rod having a sealing element coupled thereto, according to an embodiment of the disclosure.

FIG. 7 is a cross-sectional view of a distal end of a syringe, according to an embodiment of the disclosure.

FIG. 8 is another perspective view of a plunger rod having a sealing element coupled thereto.

FIG. 9 is a perspective view of a distal end of a syringe, according to an embodiment of the disclosure.

FIG. 10 is a graph depicting compliance of a prior art syringe compared to a syringe according to an embodiment of the disclosure.

FIG. 11 is a graph of test results depicting force over distance for a prior art syringe compared to a syringe according to an embodiment of the disclosure.

FIG. 12 is a graph of test results depicting flow rate over time of a prior art syringe operating in a syringe pump compared to a syringe according to an embodiment of the disclosure, for a first set of test conditions.

FIG. 13 is a graph of test results depicting flow rate over time of a prior art syringe operating in a syringe pump compared to a syringe according to an embodiment of the disclosure, for a second set of test conditions.

FIG. 14 is a graph of test results depicting flow rate over time of a prior art syringe compared to a syringe according to an embodiment of the disclosure, for a third set of test conditions.

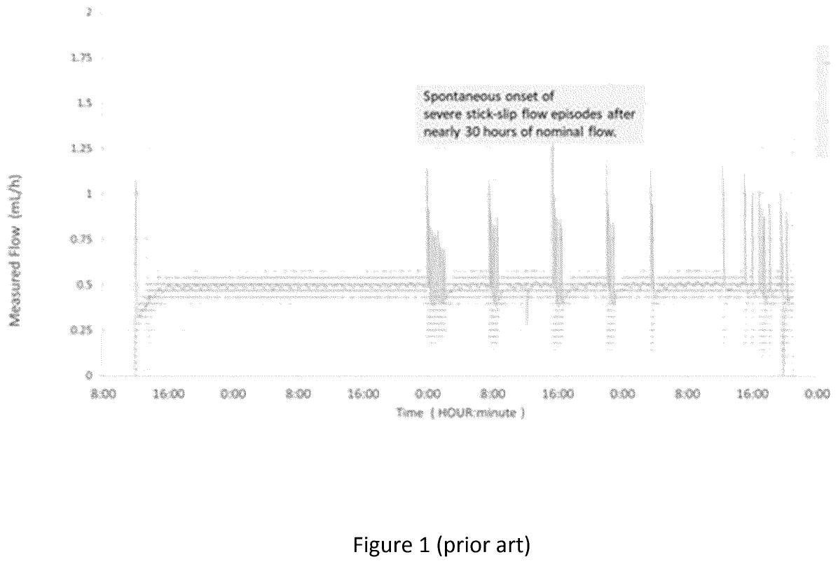

While embodiments are amenable to various modifications and alternative forms, specifics thereof have been shown by way of example in the drawings and will be described in detail. It should be understood, however, that the intention is not to limit subject matter hereof to the particular embodiments described. On the contrary, the intention is to cover all modifications, equivalents, and alternatives falling within the spirit and scope of subject matter hereof in accordance with the appended claims.

### DETAILED DESCRIPTION OF THE DRAWINGS

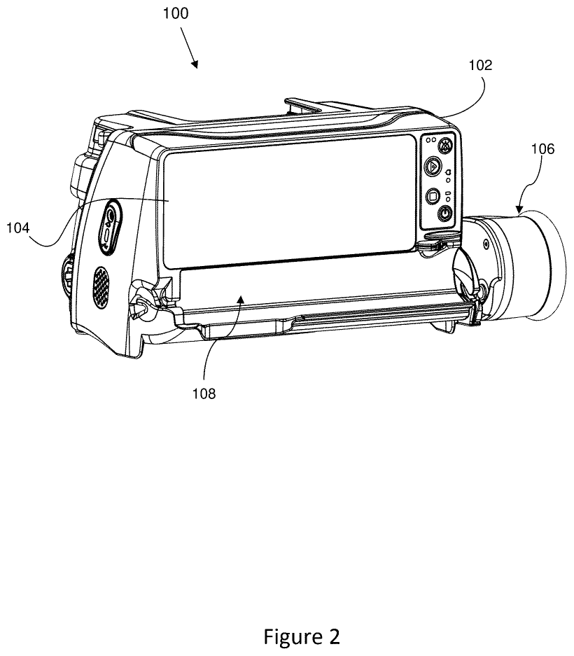

Referring now to FIG. 2, an example of a syringe pump 100 is depicted for use with embodiments of the present disclosure. Syringe pump 100 can include a housing 102, user interface 104, a syringe drive assembly 106, and a syringe receptacle 108.

The syringe drive assembly 106 can be employed for controlling delivery of a prescribed amount or dose of an infusate from a syringe that has been installed in the pump 100 (not illustrated in FIG. 2) to a patient by mechanically advancing a plunger in the syringe to deliver the infusate at a controlled rate through an infusion line fluidly connected to the syringe. In an example, a motor within pump 100 rotates a lead screw which, in turn, causes a plunger driver head assembly of the syringe drive assembly 106 to move in a direction toward the syringe receptacle 108. This movement then pushes the plunger within a barrel of the syringe located within the receptacle 108, where the barrel is held substantially in place. Moving the syringe plunger forward acts to displace a volume of infusate in the syringe outwardly from the syringe, into the infusion line, and ultimately to the patient.

In the example pump 100 of FIG. 2, the syringe receptacle 108 provides a cavity extending across the front of the syringe pump 100 such that a syringe installed therein is readily and sustainably visible. The syringe receptacle 108 is shaped and sized to accept installation of syringes of various sizes and brands therein for delivery of infusates.

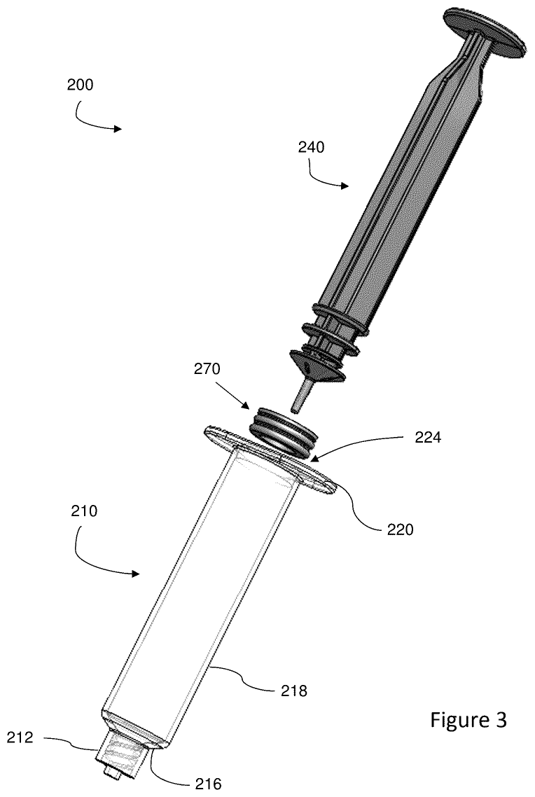

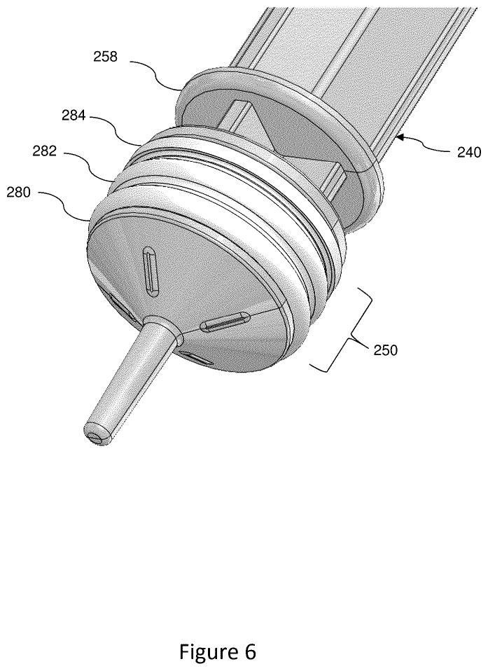

Referring now generally to FIGS. 3-9, an example of a syringe 200, according to an embodiment of the disclosure, is depicted including a barrel 210, plunger rod 240 and sealing element 270. With sealing element 270 coupled to plunger rod 240 as in FIG. 6, plunger rod 240 is operable to translate along a longitudinal axis of barrel 210 to expel infusate within barrel 210. Barrel 210 generally includes a tip 212 at a distal end, a neck 216, a body 218 and a flange 220 at a proximal end. Tip 212 includes an aperture therein, and can be configured as desired for coupling to a variety of types of extension sets, including Luer lock, enteral (including ENFit) and epidural catheter (including NRFit) tip, and other types of extension sets including those in compliance with current ISO standards. Neck 216 may be tapered as depicted in the figures, although other variations are contemplated. Body 218 of barrel 210 can be sized and shaped for a variety of common syringe capacities, for example from 1 milliliter to 100 milliliters. Flange 220 can be configured to interact with a corresponding retention feature of a syringe pump, for example to assist in securing syringe 200 in the pump during operation. At the proximal end of barrel 210 is an aperture, leading into a cavity 224 defined within barrel 210, and an inner wall 226. Cavity 224 is configured to retain infusate therein. In embodiments, a coating 228 (not labeled in the Figures) may be applied to inner wall 226. In embodiments, coating 228 may comprise a silicone oil, a non-silicone oil alternative, or other coating for lubricity. In an embodiment, coating 228 may comprise a tribofilm.

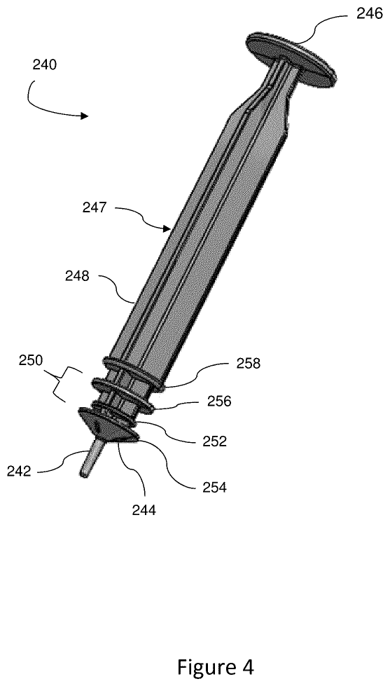

Referring also to FIG. 4, plunger rod 240 includes a substantially rigid nose 242 at a distal end, a plunger face (or stopper) 244, a press 246 at a proximal end, and a shaft 247. As generally depicted in the Figures, shaft 247 includes a plurality of ribs 248 which are arranged in a ‘+’ configuration. Other configurations of ribs 248 are also contemplated. In an embodiment, nose 242 is configured to extend into tip portion 212 of barrel 210 when the syringe is fully depressed, so as to advantageously expel any infusate in tip 212 without causing flexion of plunger face 244. Additionally, the profile of plunger face 244 can be configured to match the profile of neck 216 of barrel 210. In an embodiment, plunger face 244 may include dimpling thereon, so as to prevent or reduce inadvertent coupling between plunger face 244 and the inside of neck 216 (e.g., in the manner of a suction cup). In embodiments, the rigidity of plunger face 244 greatly reduces compliance compared to some prior approaches which may have, for example, featured elastomeric material prone to compression or expansion during use.

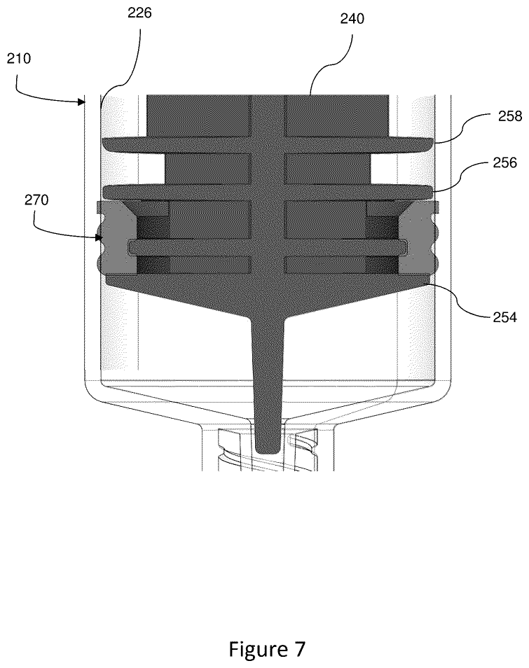

With continued reference to FIG. 4, plunger rod 240 further includes a sealing area 250, which in the embodiment depicted is adjacent plunger face 244. Sealing area 250 is configured for receipt of sealing element 270 thereupon, and generally includes an inner ring 252, a lower (or first) flange 254 and an upper (or second) flange 256. With additional reference to FIG. 7, sealing element 270 is coupleable to sealing area 250 such that sealing element 270 is bounded by lower flange 254 and upper flange 256. Inner ring 252 provides support for sealing element 250 between flanges 254 and 256. Plunger 240 further includes a reinforcing ring 258, adjacent sealing area 250. Reinforcing ring 258 may have a diameter similar to the diameters of lower flange 254 and upper flange 256. Reinforcing ring 258 is configured to limit side-to-side deflection of plunger rod 240 with respect to barrel 210, thereby reducing syringe leakage due to normal forces acting on the syringe while installed in or external to the pump, and also reducing the force required to actuate the syringe plunger.

Suitable materials for plunger rod 240 include medical grade plastics that are rigid and easy to process, such as via injection molding. One such well-known material is polycarbonate.

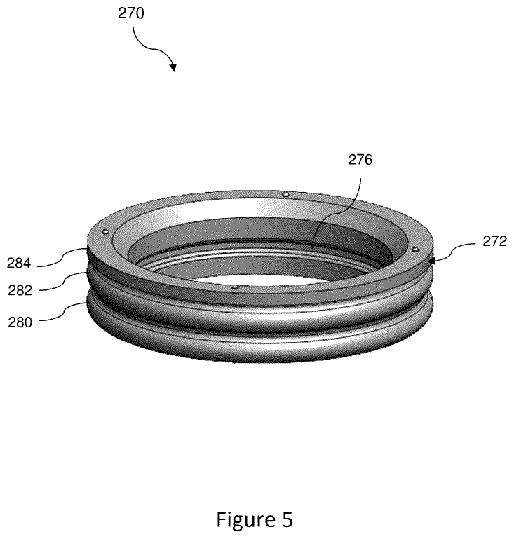

Referring now to FIGS. 5-7, sealing element 270 includes a body 272, an inner diameter 274, and an outer diameter 278. In embodiments, inner diameter 274 can include a circumferential channel 276 configured to cooperatively engage with inner ring 252 of plunger rod 240. Outer diameter 278 includes one or more seals. As depicted generally in the Figures, sealing element 270 includes a first seal 280 and a second seal 282 arranged on outer diameter 278. Seals 280 and 282 may comprise smooth surfaces, as depicted, or in other embodiments may include texturing or raised or lowered dimpling to tailor the friction characteristics of sealing element 270 as desired. Similarly, while seals 280 and 282 are depicted as being generally symmetric, it will be understood that asymmetric configurations are also contemplated as needed. Further, sealing element 270 includes a flange 284, configured to aid retention of sealing element 270 in sealing area 250 of plunger rod 240 by providing a robust surface to abut upper flange 256 of plunger rod 240. Flange 284 also supports a die cutting process to separate multiple seal elements from a compression molded sheet of elements, manufactured integrally together. Suitable materials for sealing element 270 can include isoprene, or isobutylene isoprene rubber.

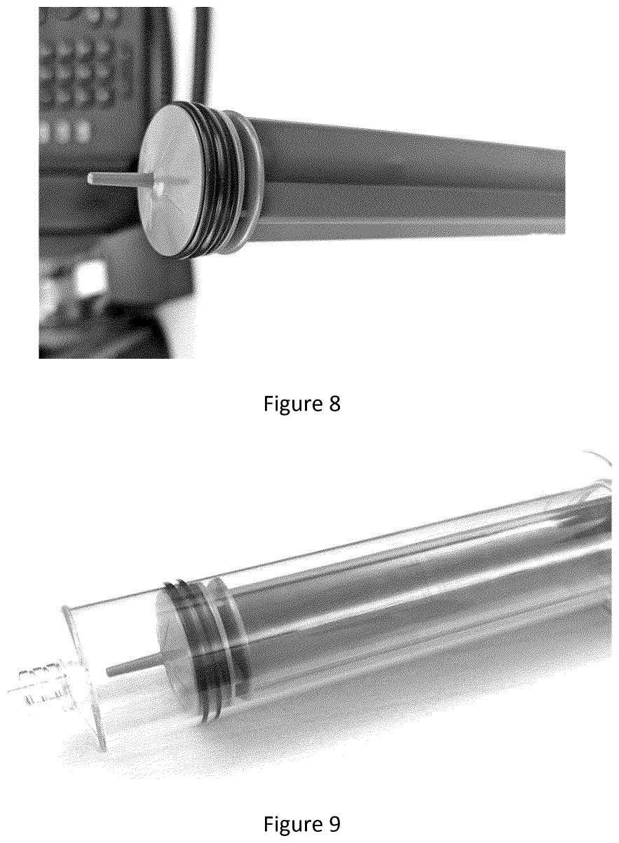

FIG. 8 depicts another perspective view of a plunger rod having a sealing element coupled thereto. FIG. 9 is a perspective view of a distal end of a syringe, according to an embodiment of the disclosure.

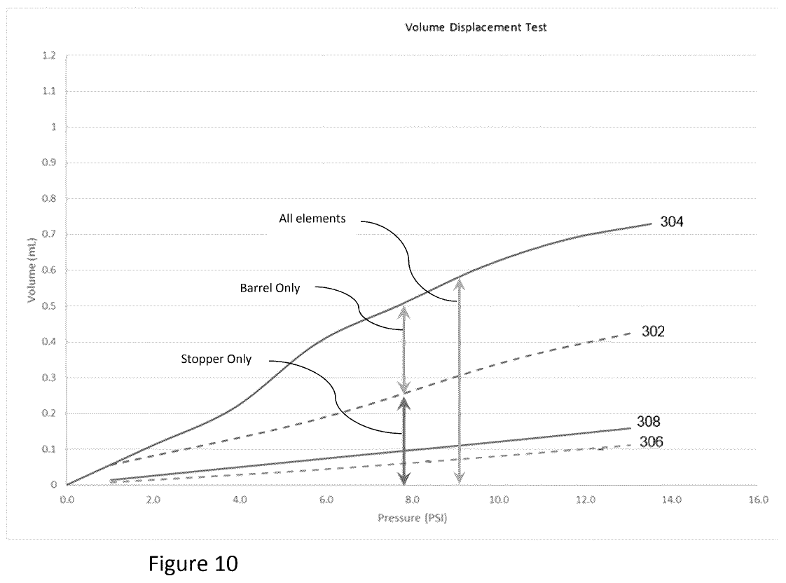

Several prototype syringes were prepared and tested in accordance with embodiments of the present disclosure. Referring now to FIG. 10, volume displacement testing was conducted of a 60 mL prior art syringe and a 60 mL improved syringe constructed in accordance with embodiments of the present disclosure. The volume displacement test is performed using a liquid filled syringe by first fixing the syringe plunger at a specific test location. A rigid, liquid filled tube is attached to the syringe, with an air pressure source connected to the end of this tube. Air pressure is increased causing the water-air boundary to be displaced toward the syringe. This displacement is measured and converted to a “displacement volume” and plotted as in FIG. 10. Persons of ordinary skill in the art will understand that “compliance” of a syringe in the context of FIG. 10 is a parameter relating displacement volume to applied pressure at a selected pressure.

Referring to FIG. 10, line 302 depicts volume displacement of a prior art syringe with its plunger/stopper located at the outlet (empty, e.g., all fluid expelled) on the (y-axis) over applied pressures (x-axis). Thus, line 302 represents volume displacement of the plunger component alone. Line 304 depicts volume displacement with the stopper/plunger at the capacity (fully extended) position, thus the difference between line 304 and line 302 represents volume displacement of the barrel component of the prior art syringe alone.

Testing was then conducted on the improved syringe as described by example herein and illustrated in FIGS. 3-9 as aforementioned. Line 306 depicts volume displacement with the plunger/stopper located at the outlet (empty). Thus line 306 represents volume displacement of the plunger component alone. Line 308 depicts volume displacement with the plunger fully extended from the barrel, thus the difference between line 308 and line 306 represents volume displacement of the barrel component alone. The improved syringe in the full position thus demonstrates an approximately eighty percent reduction in volume displacement compared to the prior art syringe. Volume displacement represents the ability of the syringe to expand to accommodate incremental fluid volume under pressure. In operation with a syringe pump, which applies pressure to the syringe plunger to displace fluid towards the patient in a controlled manner, reduced volume displacement translates to reduced start-up time as less mechanical displacement of the plunger is required to pressurize the system and reach mechanical equilibrium prior to steady state delivery. Reduced volume displacement similarly reduces time to occlusion alarm, as the syringe has a reduced capacity to absorb incremental fluid under pressure and thus reaches a threshold pressure more quickly than a syringe with higher volume displacement.

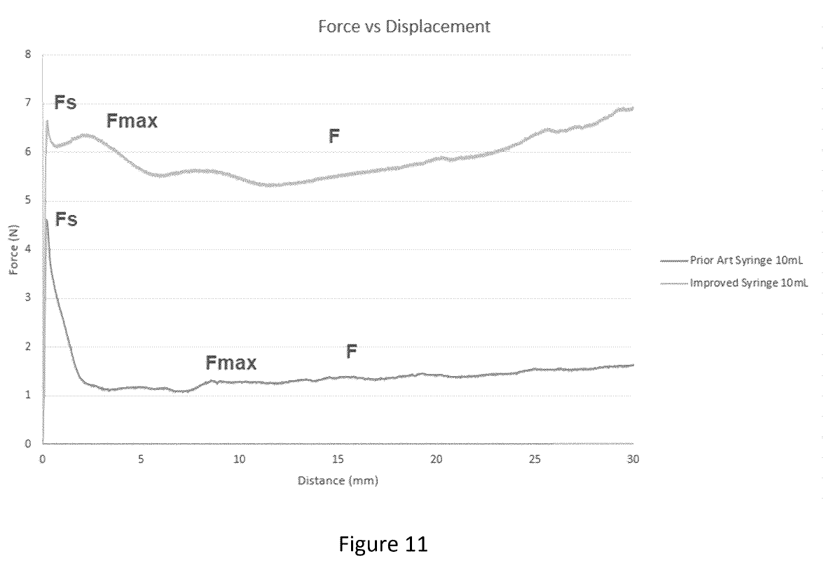

Referring now to FIG. 11, testing was also conducted to determine stick-slip behavior of a prior art syringe as well as a syringe constructed in accordance with embodiments of the present disclosure. An initial application of force to a plunger rod must overcome any static friction between the plunger seal and the inside of the barrel. The force required to overcome the static friction and allow the plunger to begin moving is represented as Fs, the force to start. After Fs, the required force briefly oscillates by dropping then rising to Fmax, the maximum force observed while the plunger is in motion. By measuring Fs, Fmax, and calculating a mean Fmean, a percentage difference between the values of each parameter can be determined. A relatively small difference between the values of these three parameters tends to result in reduced stick-slip behavior, while a larger difference between the values tends to result in increased stick-slip behavior. In FIG. 11, the x-axis represents distance and the y-axis represents force, while the upper line represents the improved syringe according to embodiments of the present disclosure and the lower line represents the prior art syringe.

Embodiments of varying syringe configurations were constructed and tested, resulting in a measured percentage difference (between Fs, Fmax and Fmean) of only one percent for a syringe constructed in accordance with embodiments of the present disclosure having a tribofilm coating applied on an inner surface of the barrel. Comparatively, a prior art syringe was also tested and the percentage difference between Fs, Fmax and Fmean was thirty-six percent. The present disclosure thus represents a significant improvement for syringe operation.

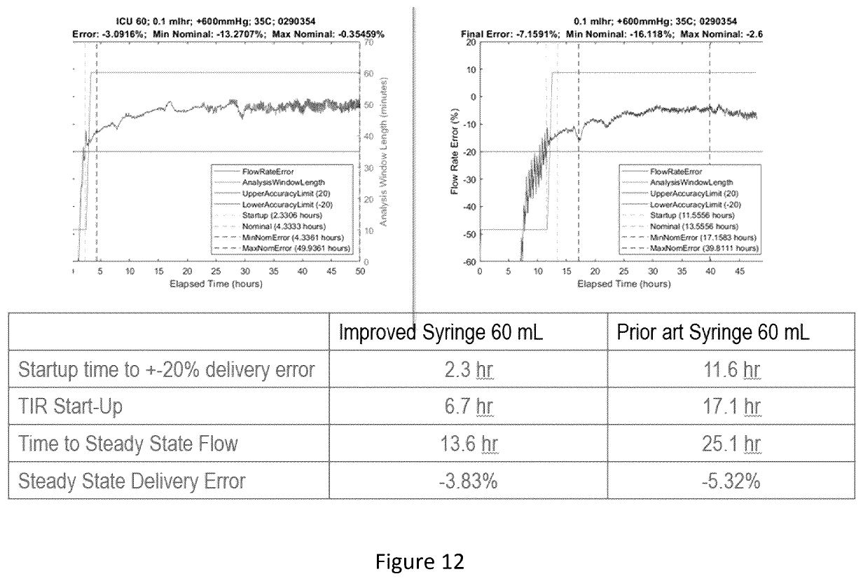

Referring now to FIGS. 12-14, each depicts test results of flow rate error for an improved syringe in accordance with embodiments of the present disclosure (left graph) compared to a prior art syringe (right graph), with notable data presented in the table. For example, the test parameters for FIG. 12 were a flow rate of 0.1 milliliters/hour, temperature of 35 C and backpressure of 600 mmHg. As is apparent from the graphs and tabular data, the improved syringe advantageously featured significantly shorter startup time, shorter time to steady state flow, and reduced steady state delivery error.

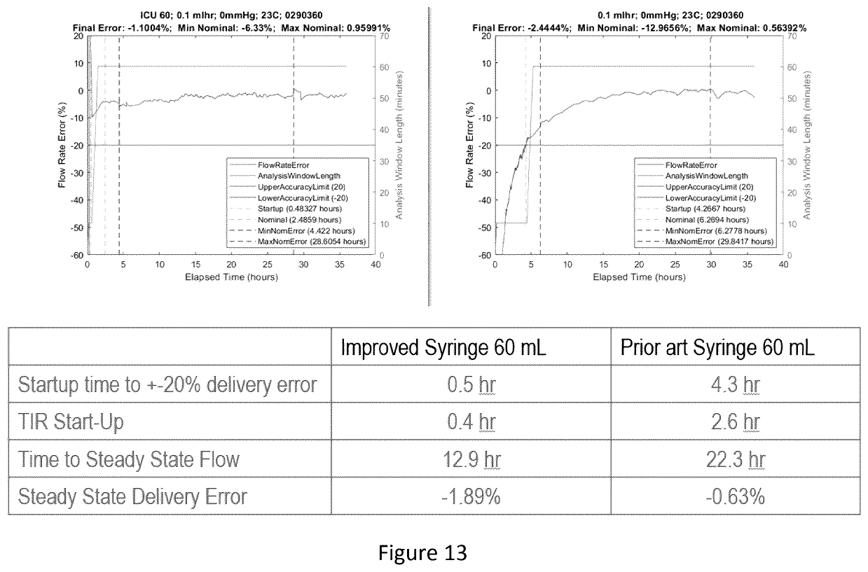

Similarly, FIG. 13 depicts results of a test conducted at a flow rate of 0.1 milliliters/hour, temperature of 23 C and backpressure of 0 mmHg. As is apparent from the graphs and tabular data, the improved syringe advantageously featured significantly shorter startup time, shorter time to steady state flow, and only marginally greater steady state delivery error.

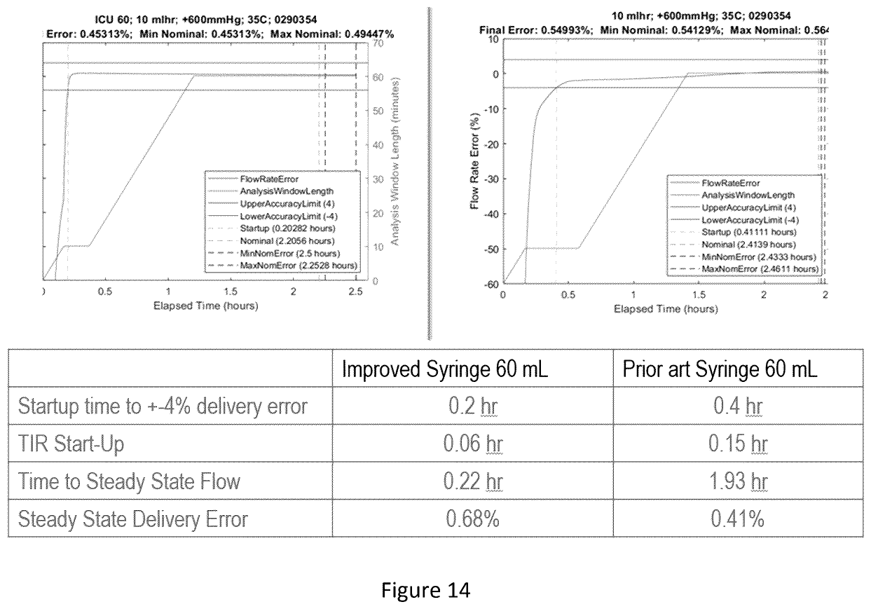

Finally, FIG. 14 depicts results of a test conducted at a flow rate of 10 milliliters/hour, temperature of 35 C and backpressure of 600 mmHg. As is apparent from the graphs and tabular data, the improved syringe advantageously featured shorter startup time, shorter time to steady state flow, and only marginally greater steady state delivery error.

Various embodiments of systems, devices, and methods have been described herein. These embodiments are given only by way of example and are not intended to limit the scope of the claimed subject matter herein. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed subject matter herein.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A syringe, comprising:
a barrel including a body, a tapered distal end extending to a tip, and a proximal end open to a cavity defined within the barrel, the cavity having an inner wall coated with a lubricious material;
a plunger rod including a shaft, a plunger face, a distal elongated nose extending from the plunger face, a proximal press portion, and a sealing area, the sealing area having a first flange, a second flange, and an inner support arranged between the first flange and the second flange; and
a sealing element having a first seal, a second seal, and an inner channel, wherein the sealing element is configured to be operably coupled with the sealing area of the plunger rod such that the inner channel of the sealing element is arranged on the inner support of the plunger rod,
wherein the plunger rod is configured to be operably coupled within the cavity of the barrel, and translatable along a length of the barrel so as to selectively expel fluid from the cavity out of the tip of the barrel, and
wherein the elongated nose of the plunger rod is configured to extend into the tip of the barrel when the plunger rod is fully depressed with respect to the barrel, so as to expel any liquid remaining in the tip without causing deformation of the plunger face.

2. A syringe, comprising:
a barrel including a body, a tapered distal end extending to a tip, and a proximal end open to a cavity defined within the barrel, the cavity having an inner wall coated with a lubricious material;
a plunger rod including a shaft, a plunger face, a distal elongated nose extending from the plunger face, a proximal press portion, and a sealing area; and
a sealing element having a first seal and a second seal, wherein the sealing element is configured to be operably coupled with the sealing area of the plunger rod,
wherein the plunger rod is configured to be operably coupled within the cavity of the barrel, and translatable along a length of the barrel so as to selectively expel fluid from the cavity out of the tip of the barrel, and
wherein the elongated nose of the plunger rod is configured to extend into the tip of the barrel when the plunger rod is fully depressed with respect to the barrel, so as to expel any liquid remaining in the tip without causing deformation of the plunger face.

3. The syringe of claim 2, wherein the sealing area of the plunger rod comprises a first flange, a second flange, and an inner support arranged between the first flange and the second flange.

4. The syringe of claim 3, wherein the sealing element further includes an inner channel, the sealing element configured to be operably coupled with the sealing area of the plunger rod such that the inner channel of the sealing element is arranged on the inner support of the plunger rod.

5. The syringe of claim 4, the plunger rod further comprising a reinforcing ring arranged on the shaft, the reinforcing ring configured to limit side-to-side deflection of the plunger rod with respect to the barrel.

6. The syringe of claim 5, wherein the first flange is adjacent the plunger face.

7. The syringe of claim 6, wherein the reinforcing ring is arranged proximal of the second flange.

8. The syringe of claim 2, wherein the tip of the barrel is configured to be coupled with an extension set.
